(19) United States
(12) Patent Application Publication
(10) Pub. No.: US 2025/0140355 A1
(43) Pub. Date: May 1, 2025

(54) FRAMEWORK FOR ANALYZING PROPERTIES OF CHEMICAL MATERIALS

(71) Applicant: MICROSOFT TECHNOLOGY LICENSING, LLC, Redmond, WA (US)

(72) Inventors: Sara Malvar MAUA, Sao Paulo (BR); Morris Eli SHARP, Seattle, WA (US); Leonardo de Oliveira NUNES, Rio de Janeiro (BR); Maria Angels DE LUIS BALAGUER, Raleigh, NC (US); Swati SHARMA, Hayward, CA (US)

(21) Appl. No.: 18/385,859

(22) Filed: Oct. 31, 2023

(51) Int. Cl. G16C 20/70; G16C 20/20; G16C 20/30; G16C 20/90

(52) U.S. Cl. CPC G16C 20/70; G16C 20/20; G16C 20/30; G16C 20/90

(57) ABSTRACT

The techniques disclosed herein enable an autonomous agent to interpret an input dataset and orchestrate a suite of software modules to perform a computational task on a representation of a chemical material. The input dataset includes a prompt defining a computational task to be performed on a chemical material. Moreover, the input dataset includes data defining a chemical included in the chemical material, molecular descriptors describing the chemical and/or the chemical material, and an external variable. The agent analyzes the benefits and drawbacks of each model within the context of the computational task to determine a technique for performing the computational task. Accordingly, the agent formulates a chain of calls invoking the functionality of data processing tools and models to perform the computational task responsive to the prompt.

### BACKGROUND

The realm of chemical research encompasses diverse disciplines such as drug discovery, materials science, chemical manufacturing processes, and energy and fuel production. A commonality among such disciplines is the expensive nature of laboratory research. That is, the time spent by a research chemist or other individual investigating and/or experimenting with chemicals and chemical formulations. Oftentimes, laboratory research represents a significant portion of both the financial and temporal costs involved in developing useful and feasible chemical products.

For instance, in the context of drug discovery, myriad challenges exist in developing new medicines as a formulation must 1) include an effective dose of therapeutics, 2) be safe for use, 3) sufficiently stable for transportation and storage, 4) comply with domestic and international regulations, and so forth. Consequently, significant effort is expended in search of formulations that satisfy all such requirements.

In another example, a research laboratory (e.g., at a university, at a research and development division of a company) in search of a new research direction may manually evaluate potential subjects of investigation through trial and error. Evaluation in this manner may often take weeks or even months. While such work is an important component of the research process, it can also be inordinately time-consuming thus restricting the productivity of said research and placing additional stress on researchers.

To alleviate these challenges, many organizations have turned to modelling and analysis of chemicals and chemical formulations through computational methods such as machine learning. However, many existing systems, while helpful in reducing some volume of lab work, may impose certain technical limitations that preclude a given system from broad applicability in diverse contexts. That is, a given system may lack the versatility and/or efficiency desired by end users. For instance, a system may require a certain quantity and/or quality of input data thus requiring additional manual effort from a research chemist or other individual to prepare input data and extract the potential benefits of said system. As such, it would be beneficial for research chemists and others to be able to perform chemical analysis and/or simulation with an automated system to streamline the research process and does not impose undue technical burdens. The following disclosure is made with respect to these and other considerations.

### SUMMARY

This disclosure describes a chemical analysis system that utilizes an automated agent to organize a suite of data processing tools and computational models to assist an end user with modelling and analysis workloads for chemical materials. That is, rather than requiring the end user to manually select and invoke individual data processing tools and computational models, the disclosed system enables the end user to simply provide an input dataset to initiate a computational task execution. For instance, the input dataset can include a natural language prompt instructing the system as to the desired actions of the end user (e.g., “predict the stability of an emulsion”). It should be understood that a chemical material can be an individual chemical or a chemical formulation comprising a combination of two or more individual chemicals.

Accordingly, the agent can interpret the input dataset to perform computational tasks as requested by the end user. As mentioned, the input dataset can include a user-generated prompt defining a computational task to be completed, data identifying a chemical included in the chemical material (e.g., an active ingredient), a molecular descriptor defining a corresponding characteristic of the chemical, and an external variable which can interact with and affect the chemical material such as water hardness.

To interpret the input dataset, the agent can be implemented as a probabilistic model such as a generative artificial intelligence (AI) application. In a specific example, the probabilistic model can be a transformer-based large language model (LLM). As such, the agent can leverage the strong language processing capability of a large language model to process the input dataset and derive an appropriate sequence of actions based on the context of the input dataset. Accordingly, the agent can support an individual input modality and/or any combination of input modalities and data types (e.g., text, image, audio).

As such, an agent utilizing a large language model such as generative pre-trained transformers (GPTs) can be uniquely effective for interpreting an input dataset where the operational context can be broad and the quantity and/or quality of available data can be inconsistent. Accordingly, the agent can determine a technique for performing the computational task defined by the prompt based on the included data (e.g., the data identifying the chemical, the molecular descriptor, the external variable) and generate the representation of the chemical material via a data processing tool.

In various examples, the representation of the chemical material can be a text string representation generated from a format provided by the user input. In a specific example, the text string representation can comply with the Simplified Molecular-Input Line-Entry System (SMILES) specification using the American Standard Code for Information Exchange (ASCII) to describe the structure of a chemical material. In this way, a given chemical material can be represented in a well-defined format to enable compatibility with various data processing tools, computational models, and so forth. For instance, the prompt received from the input dataset may describe a chemical material in plain language (e.g., “cyclohexane” or “C₁₆H₁₂”). However, a software component (e.g., a machine learning model) may only be able to understand a chemical material when provided in a specific format such as, but not limited to, SMILES. Consequently, translating the plain language text into a text string representation of the chemical material enables various software components to make use of the input provided by the user.

In addition to generating the representation of the chemical material, the agent can identify a plurality of models that can be configured by the agent to execute the technique for performing the computational task. For example, the agent can construct a list of models that, based on data availability, data quality, and the technical requirements of the computational task, can perform the computational task. In a specific example, the plurality of models can include a machine learning model, a transformer-based model, and a causal model.

Accordingly, the agent can receive a selection of a model from the plurality of models. In one example, the selection can be determined by the agent itself. By leveraging the analytical capabilities of the agent, it can determine the respective benefits and drawbacks of each of the plurality of models in relation to the computational task and generate a ranked list of the models. Consequently, the agent can select one or several top ranked models from the ranked list to perform the computational task. In another example, the agent may simply identify the eligible models and provide a listing of the models to a user (e.g., a research chemist) for a final selection. Given a selection of a model, the agent can invoke the functionality of the model to execute the computational task. The resultant output of the selected model can thus be provided to the user as a response to the prompt.

In contrast to existing approaches, the disclosed system can, through the agent, orchestrate a suite of tools and computational models to automate the analysis and/or simulation of chemical materials. Stated another way, the techniques discussed herein enable a system to interpret an input dataset and automatically construct a chain of calls to various data processing tools, computational models, and so forth to perform a computational task. Conversely, many existing approaches place the burden of data preparation, model selection, programming, and other tasks on the end user thus creating a potentially untenable workload. These technical challenges can be further exacerbated when an end user is unfamiliar with some computational tools and must then expend additional effort to learn those tools to even determine whether a given tool is potentially useful. In this way, the disclosed system streamlines the process of chemical analysis and simulation thereby improving laboratory efficiency while also enhancing the useability and accessibility of powerful computational tools.

In another example of the technical benefit of the present disclosure, utilizing an automated agent to organize a suite of data processing tools, computational models, and the like provides an improved user interface enabling simplified execution of computational tasks. As mentioned, many existing approaches require extensive manual effort to prepare input data and program a model to perform a computational task. Oftentimes, such approaches involve myriad data formats, programming languages, user interfaces and so forth leading to an increased risk of confusion and error. Conversely, utilizing the techniques described herein, an end user interacts with the automated agent using natural language to perform computational tasks with minimal data preparation and other extraneous tasks.

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. The term “techniques,” for instance, may refer to system(s), method(s), computer-readable instructions, module(s), algorithms, hardware logic, and/or operation(s) as permitted by the context described above and throughout the document.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 is a diagram illustrating an automated agent performing a computational task in response to a user-generated prompt.

FIG. 2 is a diagram illustrating a framework orchestrated by an agent for performing a computational task on a representation of a chemical material.

FIG. 3 is a diagram illustrating a control flow of a framework orchestrated by an agent selecting a model to perform a computational task on a representation of a chemical material.

FIG. 4 is an architecture of an agent and accompanying modules that implement a framework for performing a computational task on a representation of a chemical material.

FIG. 5 is a flow diagram showing aspects of a process for interacting with an agent to perform a computational task on a representation of a chemical material.

FIG. 6 is a flow diagram showing aspects of an additional process for interacting with an agent to perform computational tasks on a representation of a chemical material.

FIG. 7 is a computer architecture diagram illustrating an illustrative computer hardware and software architecture for a computing system capable of implementing aspects of the techniques and technologies presented herein.

FIG. 8 is a diagram illustrating a distributed computing environment capable of implementing aspects of the techniques and technologies presented herein.

### DETAILED DESCRIPTION

This disclosure provides a system and technique for using an automated agent to orchestrate a suite of data processing tools and computational models to enhance the versatility and efficiency in modeling and analysis of chemical materials. As mentioned above, the agent can be a generative probabilistic model and can be implemented using a large language model (LLM). As such, the agent can interpret a user-generated prompt defining a computational task to derive context and technical requirements. While the prompt may be formatted as a natural language text input, it should be understood that the agent can be a multimodal model and process any suitable input data type (e.g., text, image, audio).

Moreover, the agent can further learn the context and technical requirements of the computational task based on data included with the prompt as part of an input dataset. For instance, the input dataset can include data identifying a chemical such as a common name, a chemical structure, a database identification number, and the like. In one example, the identified chemical may be a particular chemical of interest that an end user plans to investigate. Accordingly, by performing various computational tasks in collaboration with the agent, the end user can gain insight on the chemical of interest and form a research direction that would have otherwise required significant manual experimentation.

The system as described herein can be implemented in accordance with a software framework that provides a standardized software architecture for constructing applications utilizing a particular tool. In a specific example, the present system can utilize the LangChain framework to configure a generative probabilistic model (e.g., a large language model) as an agent to orchestrate a suite of data processing tools, computational models, data sources, and so forth. Another example of such a framework is the SEMANTIC KERNEL by MICROSOFT which likewise provides a standardized software architecture for implementing generative probabilistic model functionality in various applications.

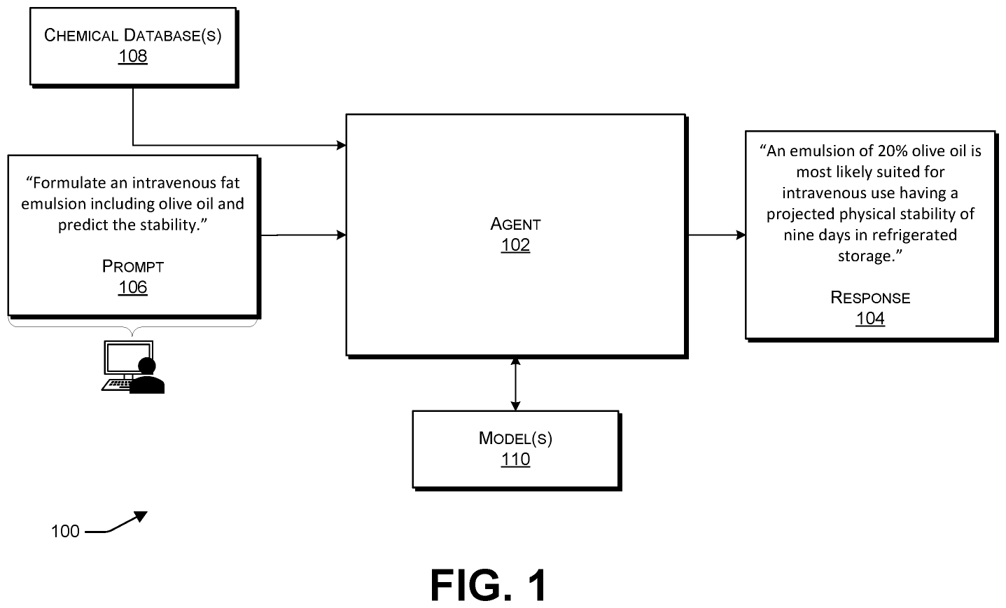

FIG. 1 illustrates a system 100 in which an agent 102 generates a response 104 pursuant to a user-generated prompt 106 submitted as a query to the agent 102. An end user (e.g., a research chemist) provides the prompt 106 as an instruction for the agent 102 to accomplish. In the present example, the prompt 106 instructs the agent 102 to “formulate an intravenous fat emulsion including olive oil and predict the stability.” Intravenous fat emulsions are a well-known tool to provide nutritional support to patients who are unable to incorporate a sufficient amount of fat in their diet.

Accordingly, the agent 102 can derive several pieces of information from analyzing the prompt 106. Firstly, the agent 102 can determine a computational task involving developing a chemical material (an emulsion) and predicting the stability of the chemical material (the ability to resist changes in physicochemical properties over time). Secondly, the agent 102 can extract data identifying a chemical that is included in the chemical material. In this example, the data identifying the chemical is a common name (olive oil). Thirdly, the agent 102 can determine an external variable that can affect a property of the chemical material as well as introduce certain constraints on the chemical material. In the present example, the external variable is the knowledge that chemical material is to be used intravenously. Consequently, the agent 102 is constrained by this external variable to generate definitions of chemical materials that are safe for use in a circulatory system. As mentioned above, a chemical material can be a single chemical or a chemical formulation comprising a combination of two or more individual chemicals.

It should be understood that while the prompt 106 can include some components of an input dataset (e.g., the computational task, the data identifying the chemical, the external variable), portions of the input dataset can also originate from sources other than an end user. For example, while the prompt 106 provides a common name for “olive oil,” performing the computational task may require one or several technical representations of “olive oil” (e.g., chemical formula, chemical properties). Accordingly, the agent 102 can be configured with a connection to a chemical database 108 such that the agent 102 can extract and utilize chemical data such pertaining to “olive oil” as identified by the prompt 106. For instance, the agent 102 can obtain chemical descriptors from the chemical database 108. In various examples, the chemical descriptors can be numerical values quantifying properties of a chemical (e.g., electronic, geometric, steric properties). Moreover, in the context of the present disclosure, the agent 102 can be configured to autonomously retrieve data such as the chemical descriptors in accordance with the computational task defined by the prompt 106. In a specific example, the agent 102 generates a data request (e.g., an application programming interface (API) call) which is accordingly transmitted to a database. In response, the database provides the data specified by the request.

Subsequently, the agent 102 can proceed to generate a definition of a chemical material (e.g., a chemical formulation) for an intravenous fat emulsion in accordance with the computational task defined by the prompt 106. As such, the agent 102 can identify a plurality of models 110 which can be configured by the agent to accomplish this task. For example, the plurality of models 110 can include a machine learning model (e.g., an artificial neural network, a support vector machine), a transformer-based model, and a causal model. In a specific example, the causal model is a Bayesian network that represents a set of variables and their conditional dependencies in a directed acyclic graph comprising a set of vertices connected by directed edges (i.e., arcs). Accordingly, a Bayesian network can be amenable for analyzing an event that has occurred and predicting the likelihood that any one of several possible causes contributed to the occurrence of the event. For instance, given a set of symptoms, a Bayesian network can be used to compute the probabilities of various diseases.

As will be discussed further below, the agent 102 can evaluate the benefits and draw backs of each of the available models 110 within the context of the computational task at hand (generating a chemical material) and the input data (e.g., data quantity, data quality). Based on this evaluation, the agent 102 can select a model from the plurality of models 110 to perform the computational task. For instance, the agent 102 may determine for the present example that, given an availability of accurate chemical descriptors from the chemical database 108, an artificial neural network implementing a quantitative structure-property relationship (QSPR) model represents an ideal approach for generating a chemical material for an intravenous fat emulsion of olive oil.

It should be understood that a chemical formulation, as discussed herein, can be defined by an identity of the chemicals that form the chemical formulation and the relative amount of each chemical. Consequently, generating a chemical formulation involves selecting the chemicals, calculating the relative amounts, and producing a representation of the chemical formulation (e.g., text, image). In addition, while the agent 102 can autonomously select the model from the plurality of models 110, any suitable method of model selection can also be utilized. For instance, the agent 102 may provide a model recommendation to an end user based on an analysis of the plurality of models 110. Accordingly, the end user can leverage subject matter expertise and/or past experience with various models to form a final selection. Moreover, as used herein, “quality” and “data quality” refer to a state of preparation and/or information density of input data. In a specific example, a sparse matrix can be considered a different quality of data in relation to a dense matrix. In another example, a dataset that is labeled for processing by an artificial intelligence can be considered a different quality of data in relation to unlabeled data.

As the computational task defined by the prompt 106 of the present example comprises two operations (generating a chemical material and predicting the stability of the chemical material), the agent 102 can select a same or different approach for predicting the stability as generating the chemical material. For instance, the agent 102 may determine that the model selected for generating the chemical material is also the most suitable approach for performing a subsequent analysis on the chemical material. In an alternative example, the agent 102 may determine that a different approach would most likely yield ideal performance. For example, while a QSPR model can be utilized to generate the definition of the chemical material, the agent 102 can determine that a different quantitative structure-activity relationship (QSAR) model is ideal for predicting physicochemical properties such as the stability of an emulsion.

Accordingly, the agent 102 can process the outputs of the models 110 to format a response 104. In various examples, the models 110 may generate outputs that are not intuitively readable (e.g., raw numerical data) which, in the absence of the agent 102 may require expertise to interpret. However, the agent 102 can process the output data to provide a readily understandable response 104 stating that “an emulsion of 20% olive oil is most likely suited for intravenous use having a projected stability of nine days in refrigerated storage.” While the present response 104 can be formatted as a natural language text response, it should be understood that the response 104 can be output in any suitable format (e.g., image, text, audio). Moreover, it should be understood that the response 104 is merely an example and is illustrated for readability and brevity. In practice, a definition of a chemical material (e.g., a chemical formulation) provided in a response 104 may include a comprehensive listing of chemicals and associated relative amounts.

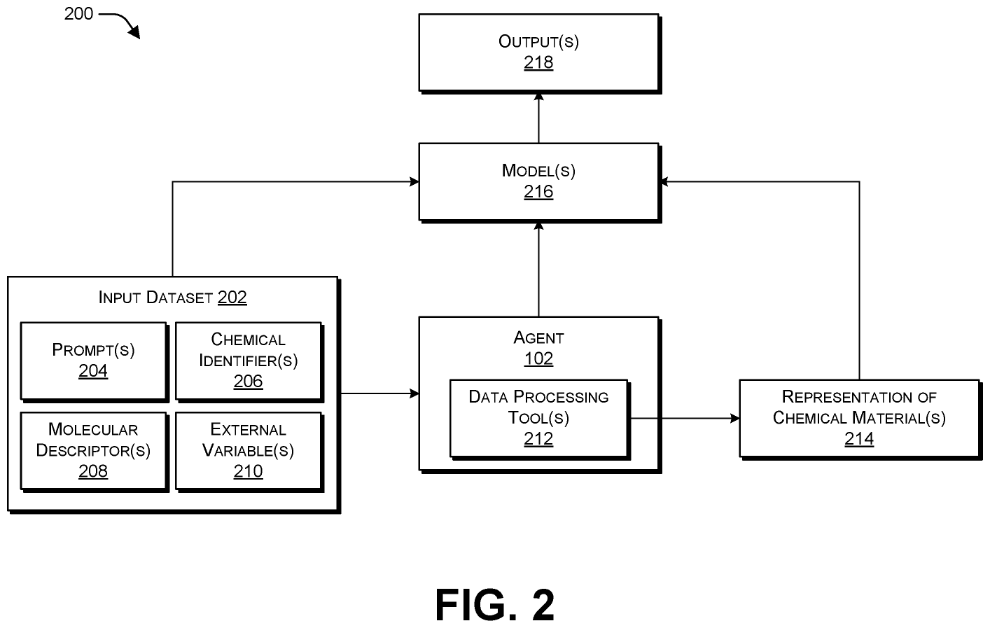

FIG. 2 illustrates aspects of a demonstrative architecture 200 of the system 100 introduced in FIG. 1. In the illustrated example, the agent is implemented as a cloud-based service that is physically remote from an end user device used for submitting an input dataset 202. As mentioned above, the input dataset 202 includes a prompt 204 defining a computational task such as the prompt 106 discussed above with respect to FIG. 1. In addition, the input dataset 202 includes a chemical identifier 206 comprising data identifying a chemical in a chemical material. The chemical identifier 206 can be a common name, a chemical structure, a database ID number, and the like. As used herein, a chemical is any single substance having a defined composition (e.g., dimethyl carbonate). A chemical can also be an organic molecule including biological molecules such as proteins and polynucleotides.

Furthermore, the input dataset 202 includes a molecular descriptor 208 comprising data quantifying a characteristic of the chemical specified by the chemical identifier 206 such as molar refractivity, polarizability, and so forth. Finally, the input dataset 202 includes an external variable 210 defining an environmental factor that can affect a property of a chemical material such as water hardness. As such, external variables 210 bear consideration when generating chemical materials and performing subsequent computing tasks. As mentioned, the input data 202 can originate wholly from an end user via a user device (e.g., a laptop, a desktop computer) with the end user providing the prompt 204 and all requisite data 206-210. Alternatively, the input dataset 202 can originate from a combination of the end user and other sources such as a public chemical database, a private laboratory repository, a shared data storage service, and the like. While the examples discussed herein relate to individual components (e.g., a prompt 204, an external variable 210, a data processing tool 212), it should be understood that any number of components can be utilized to implement the described techniques. For instance, the input dataset 202 can include multiple prompts, multiple chemical identifiers 206, and so forth.

The input dataset 202 is then provided to the agent 102 for interpretation. That is, the agent extracts and analyzes the constituent components of the input dataset 202 to learn a computational task to be performed. As discussed above, the agent 102 analyzes the prompt 204 to determine the context and technical requirements of the computational task and devises a technique for performing the computational task. Accordingly, the agent 102 can utilize a data processing tool 212 to generate a representation of a chemical material 214 in accordance with the computational task defined by the prompt 204. In various examples, the representation of the chemical material 214 can be a text string representation utilizing a standard specification to represent chemical structures.

For instance, the representation of the chemical material 214 can utilize the Simplified Molecular-Input Line-Entry System (SMILES). In an alternative example, the representation of the chemical material 214 can be a Self-Referencing Embedded String (SELFIES). In still another example, the representation of the chemical material 214 can be a pictorial representation generated from a text string representation. That is, the agent 102 calls upon a first data processing tool to generate a text string representation of the chemical material 214 and subsequently calls upon a second data processing tool to convert the text string representation to a pictorial representation.

Considerations as to the format of the representation of the chemical material 214 can be incorporated by the agent 102 when selecting a model 216 to perform the computational task. For example, the selected model 216 may be compatible with a pictorial representation of chemical material 214 and not a text string representation. In this way, the agent 102, via a suite of data processing tools 212, can autonomously prepare data received from the input dataset 202 for compatibility with various models 216. Stated another way, the agent 102 can independently interpret the input dataset 202 to determine a technique for performing a computing task comprising a chain of calls to a data processing tool 212 and a model 216.

In a specific example, the input dataset 202 comprises a prompt 204 instructing the agent 102 to “predict the stability of an oil in water emulsion containing sodium alginate.” To perform this task, the agent 102 generates a chain of calls as mentioned above. The chain of calls includes a first call to a chemical database to retrieve molecular descriptors 208 relating to the prompt 204 (e.g., sodium alginate). Subsequently, a second call in the chain of calls invokes a data processing tool 212 to generate a representation of the chemical material 214 (i.e., the “oil in water emulsion containing sodium alginate”). In addition, the agent 102 determines a model 216 that is most suitable for performing the current computational task. Consequently, a third call in the chain of calls initializes and/or trains the model 216 to perform a prediction task. Finally, a fourth call in the chain of calls provides the representation of the chemical material 214, any additional data such as the external variables 210, and invokes the model 216 to execute the computational task defined by the prompt 204. In this way, the agent 102 autonomously devises and executes a technique for performing the computational task by invoking various components of the architecture 200.

In one example, calling the model 216 comprises processing or otherwise altering some or all of the input dataset 202 through the agent 102 and/or a data processing tool 212 under control of the agent 102. That is, the model 216 receives an input from the agent 102 such as the representation of the chemical material 214. In an alternative example, the model 216 can be configured to directly receive some or all of the input dataset 202. Stated another way, by configuring the 216 to perform the computational task defined by the prompt 204, the agent 102 grants the model 216 access to the input dataset 202 which the model 216 operates upon.

Accordingly, the model 216 generates an output 218 in response to an invocation (e.g., a call) by the agent 102 to perform the computational task. The output 218 can be any form of computational output that is responsive to the prompt 204. In one example, the prompt 204 instructs the agent 102 to predict the solubility of a chemical material. In response, the output 218 is a numerical volume in accordance with the computational task defined by the prompt 204. In another example, the prompt 204 instructs the agent 102 to calculate a definition for an experimental chemical material (e.g., a chemical formulation). Accordingly, the output 218 can be a text output identifying the constituent chemicals of the chemical material and relative amounts for each chemical. Moreover, the output 218 can include an image output illustrating a molecular structure of the experimental chemical material.

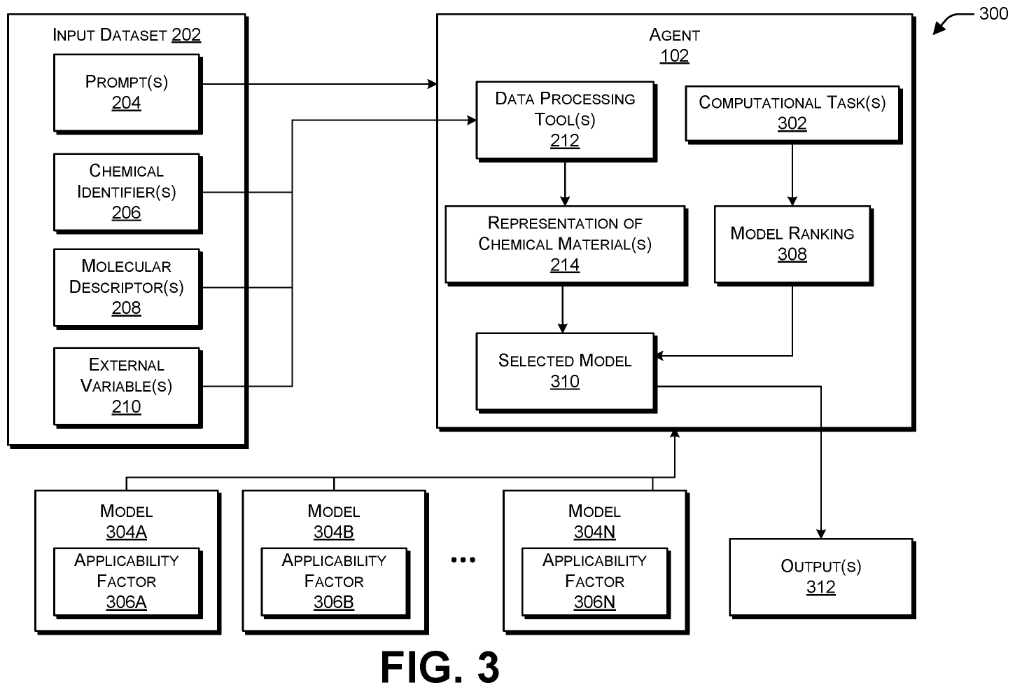

FIG. 3 shows aspects of a further illustrative architecture 300 of the system 100 introduced in FIG. 1. As discussed above, an agent 102 is configured to receive an input dataset 202 comprising a prompt 204, a chemical identifier 206, a molecular descriptor 208, and an external variable 210. In the present example, the agent 102 interprets the natural language prompt 204 to determine a computational task 302 (e.g., “predict the stability of an emulsion”). In determining the computational task 302, the agent 102 is configured to define specific characteristics and technical requirements of a goal specified by the prompt 204. That is, the agent 102 determines the nature of the computational task 302 (e.g., regression, classification, clustering, anomaly detection, recommendation) and the techniques for performing the computational task 302 given a various characteristics of the input dataset 202 such as a data format of the chemical identifier 206, the molecular descriptors 208, and/or the external variable 210, the quantity of available data, the quality of available data, and so forth. In addition, the agent 102 utilizes a data processing tool 212 to generate a representation of a chemical material 214 which can be operated upon by computational tools.

As discussed above, the agent 102 is configured to identify a plurality of models 304A-304N that are capable of being configured by the agent 102 to perform the computational task 302. For instance, the agent 102 may identify a machine learning model 304A, a transformer-based model 304B, and a causal model 304N as suitable for performing the computational task 302. In an alternative example, the agent 102 can be configured with a predetermined set of models 304A-304N selected by an external entity (e.g., a computational chemist, a system engineer).

It is well known that differing models 304A-304N each present certain benefits and draw backs. For example, a classical machine learning model can benefit from short training times in relation to other alternative models and achieve high prediction accuracy through optimization techniques such as gradient boosting. However, drawbacks include high demands for labor-intensive data preparation (e.g., cleaning, feature engineering) as well as retraining to support new contexts (e.g., a new chemical).

In another example, a transformer-based model can address some of the draw backs of machine learning models by eliminating a reliance on certain types of data (e.g., molecular descriptors 208) while also requiring minimal data preprocessing (e.g., cleaning). Moreover, a transformer-based model can incorporate support for new contexts with minimal retraining. However, transformer-based models achieve these benefits by trading off for relatively longer training times and reduced prediction accuracy in comparison to machine learning models. Generally described, a transformer-based model is a deep learning architecture that relies on a multiheaded attention mechanism to mimic cognitive attention by calculating “soft” weights for individual tokens of an embedding. In contrast to “hard” weights, a “soft” weight can be recalculated during runtime to provide flexibility during operation. In this way, a transformer-based model can maintain awareness of context (e.g., when processing a string of tokens) leading to improved processing and training times.

Accordingly, the agent 102 can be preconfigured with knowledge of the benefits and drawbacks of each of the plurality of models 304A-304N (e.g., a corpus of artificial intelligence research). Consequently, the agent 102 can incorporate a cost/benefit analysis when determining a technique for performing the computational task 302. As shown in FIG. 3, each model 304A-304N is assigned a corresponding applicability factor 306A-306N which serves to quantify an ability of the associated model 304A-304N to execute the technique for performing the computational task 302. For example, an abundance of molecular descriptor data 208 and pre-prepared chemical identifier data 206 in the input dataset 202 can result in an elevated applicability factor 306A for a machine learning model 304A as opposed to an applicability factor 306B for a transformer-based model 304B as such conditions particularly suit the characteristics of a machine learning model 304A. In another example, a lack of available chemical identifiers 206 or robust preprocessed input data can result in a depressed applicability factor 306A for a machine learning model 304A and an elevated applicability factor 306B for a transformer-based model 304B.

In context of the present disclosure, the “ability” of a given model 304A-304N to execute a technique for performing a computational task 302 can be a consideration of the technical benefits and draw backs of each model 304A-304N within the context of a specific computational task 302, available data, data quality, and so forth. That is, the applicability of a given model 304A-304N. For instance, the architecture of a machine learning model can be more generally amenable to performing classification tasks in relation to the architecture of a causal model. Hence, the machine learning can be said to possess a greater “ability” to perform a classification task than a causal model. In another example, a computational task 302 may involve an experimental or otherwise obscure chemical. Consequently, molecular descriptors for the chemical may be unavailable. As such, models that rely upon molecular descriptors for known chemicals such as a machine learning model trained on a specific set of chemicals can be said to be lack the “ability” to perform the computational task 302 for a chemical that is outside of its training set.

Subsequently, the agent 102 generates a model ranking 308 of the plurality of models 304A-304N comprising a ranked list in which the models 304A-304N are ordered based on the corresponding applicability factors 306A-306N. As such, the agent 102 can designate a selected model 310 to perform the computational task 302. In various examples, the selected model 310 can be a top-ranked model from the model ranking 308. That is, the selected model 310 possesses the highest applicability factor 306A among the models 304A-304N of the model ranking 308. The selected model 310 is then configured by the agent 102 with the representation of the chemical material as well as some or all of the input dataset 202 such as the chemical identifier 206, the molecular descriptor 208, and/or the external variable 210. Accordingly, the selected model 310 generates an output 312 upon completion of the computational task 302 that is responsive to the prompt 204.

In various examples, configuring the selected model 310 to perform the computational task 302 involves training the selected model 310 to exhibit a desired behavior with respect to the technical characteristics of the computational task 302. In a specific example, the computational task 302 is to predict the solubility of a chemical material. Accordingly, the agent 102 configures the selected model 310 to predict solubility by training the selected model 310 on a predetermined dataset of known values. That is, the predetermined dataset comprises definitions of various chemical materials (e.g., molecular descriptors) and corresponding solubilities. Under supervision of the agent 102, the selected model 310 iteratively learns to maximize the accuracy of predictions by analyzing patterns in the predetermined dataset with the agent 102 optimizing parameters of the selected model 310 based on the input data and a desired outcome. As such, the selected model 310 can be tasked with analyzing a previously unencountered chemical material (e.g., the representation of the chemical material 214) and produce an accurate prediction.

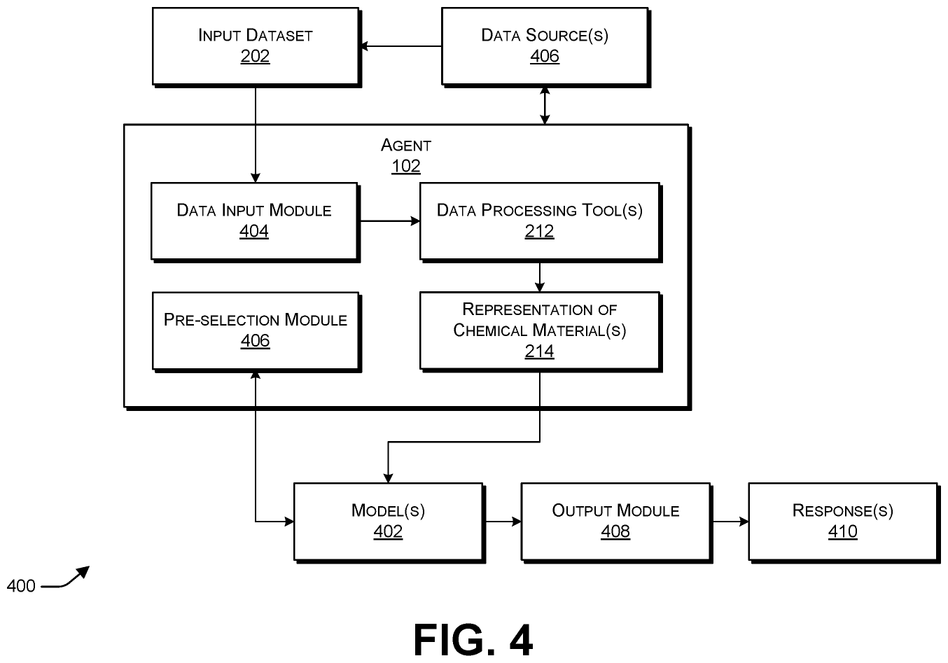

FIG. 4 demonstrates aspects of an illustrative software architecture 400 that incorporates an agent 102 to orchestrate a suite of software components such as a data processing tool 212 and a model 402. As mentioned, the agent 102 can be implemented as a generative probabilistic model such as a large language model. Examples of large language models include GPT-4 by OPENAI, LAMDA by GOOGLE, and LLAMA by META. Any of these existing models or other models that offer similar functionality can be adapted for use with the systems of this disclosure.

The architecture 400 includes a data input module 404 which is configured to retrieve data from various data sources 406. The data sources 406 can include the input dataset 202, publicly available databases (e.g., research institutions, universities), as well as private databases such as a repository of experimental data belonging to an individual user and/or a laboratory associated with the agent 102. The agent 102 may select one or more data sources 406 (e.g., external databases) to use for performing to the computational task. The data input module 404 is responsible for preprocessing and normalizing data retrieved from the data sources 406. In this way, the data input module 404 ensures data compatibility with other modules of the architecture 400. In one example, the data input module 404 can be a component of the agent 102 and thus under control of the agent 102. In another example, the data input module 404 is a standalone module which the agent 102 calls upon to receive and process input data. For instance, the data input module 404 can be configured to operate with various application programming interfaces (APIs) to provide the agent 102 access to a variety of data sources 406.

In addition, the architecture 400 includes a data processing tool 212. As discussed above, the data processing tool 212 generates representations of chemical materials 214 (e.g., a text string representation) from input data. As with the agent 102, the data processing tool 212 can be implemented using a generative probabilistic model such as a large language model. By leveraging the strong language capabilities of probabilistic models, the data processing tool 212 generates accurate and concise representations of chemical materials 214 based on the input data. In a specific example, an input dataset includes data identifying a chemical to be included in a chemical material and a molecular descriptor defining a desired characteristic of the chemical material. Accordingly, the data processing tool 212 generates a representation of the chemical material 214 satisfying these above requirements. It should be understood that the data processing tool 212 can be the same or a different generative probabilistic model as the agent 102.

In addition, the data processing tool 212 can be configured to generate embeddings from input data. An embedding is a numerical representation of non-numerical information (e.g., natural language text, images) For instance, an input dataset may include incomplete or unformatted information regarding a chemical and/or chemical material such as a natural language text description of a chemical property, a drawing of a chemical structure, and so forth. That is, various components of the input dataset can be formatted in different data types. In response, the data processing tool 212 generates a numerical representation of the information such that the information can be processed by (i.e., compatible with) other modules of the architecture 400 such as another data processing tool, the agent 102, and/or the model 402. In this way, the architecture 400 can provide support for and/or operate upon any type of input data irrespective of formatting and/or data type.

The architecture 400 further comprises a pre-selection module 406 which is configured to identify a plurality of models that are capable of being configured by the agent 102 to perform a given computational task. In a specific example, the pre-selection 406 is configured to compute a plurality of applicability factors and generate a model ranking as described above with respect to FIG. 3. Moreover, the pre-selection module 406 can also be configured to select one or a series of models 402 from the plurality of models to perform the computational task. Conversely, the agent 102 and/or an end user can perform the final selection of the model 402.

Accordingly, the agent 102 trains the model 402 to perform a given computational task as described above with respect to FIG. 3. The computational task can be defined by a user-generated prompt. For instance, a prompt may instruct the agent 102 to predict a property of a chemical material (e.g., the stability of an emulsion). Once the model 402 is trained, the representation of the chemical material 214 is provided to the model 402 along with any pertinent input data such as molecular descriptors, external variables, and so forth.

In conjunction with the model 402, the architecture 400 includes an output module 408 to generate a human-readable and interpretable response 410. As mentioned above, a model 402 may generate outputs that are not intuitively readable (e.g., raw numerical data) which can require prior knowledge or expertise to understand. In other situations, the output may simply not be human-readable. As such, the output module 408 is configured to translate an unformatted model output into a human-readable response 410. In a specific, the model 402 produces a series of numerical values in response to a computational task to predict several properties of a chemical material where each numerical value is associated with an identification number. Accordingly, the output module 408 reformats the numerical values as a response 410 that uses plain language identifiers (e.g., solubility, stability, lipophilicity). Furthermore, the output module 408 can generate responses 410 in any suitable format such as an image, a block of text, and so forth.

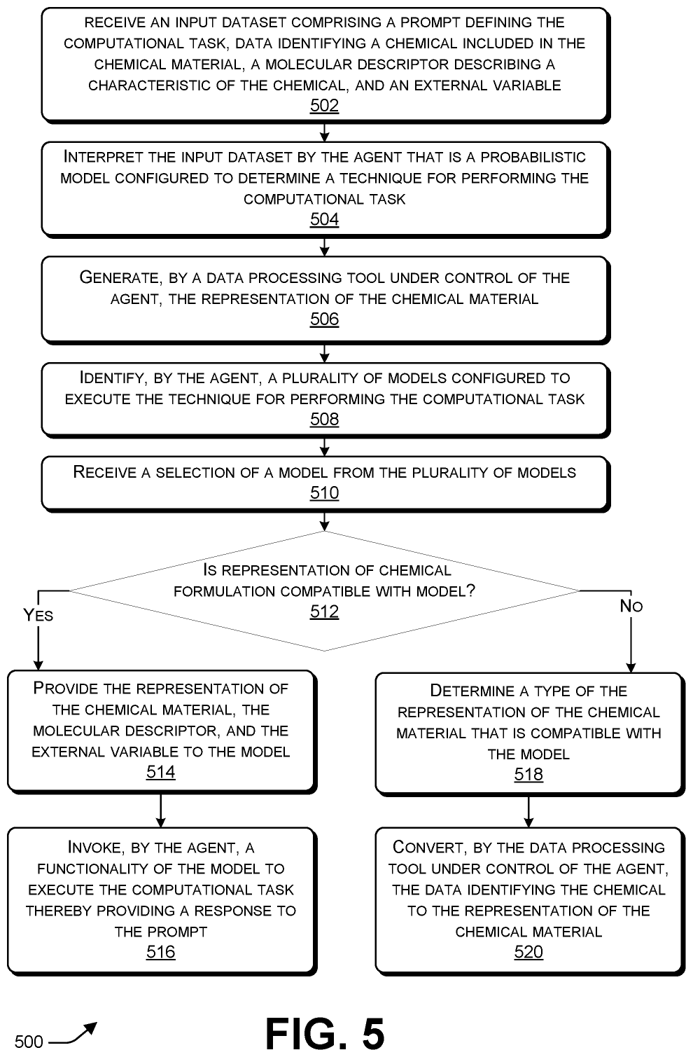

FIG. 5 illustrates aspects of a process 500 for interacting with an agent to perform a computational task on a representation of a chemical material. The process 500 may be implemented with any of the architectures and frameworks shown in FIGS. 1-4 or with alternative architectures and frameworks. With respect to FIG. 5, the process 500 begins at operation 502 in which a system receives an input data comprising a prompt defining the computational task, data identifying a chemical included in the chemical material, a molecular descriptor describing a characteristic of the chemical, and an external variable. As discussed above, the prompt can be a natural language statement instructing the system as to the desired actions of the end user (e.g., “predict the stability of an emulsion”). Moreover, the input dataset can be assembled from a plurality of data sources including a user device, a public database, a private repository, and the like.

Next, at operation 504, the system interprets the input dataset by the agent that is a probabilistic model configured to determine a technique for performing the computational task. As mentioned, examples of the probabilistic model include generative artificial intelligence models such as large language models. Unlike other artificial intelligence models, such as recurrent neural networks and long short-term memory (LSTM) models, transformer-based large language models make use of a native self-attention mechanism to identify vague context from limited available data and even synthesize new content from images to code.

Proceeding to operation 506, the system generates, by a data processing tool under control of the agent, the representation of the chemical material. As described above, the representation of the chemical material can be a text string representation in accordance with various standards such as SMILES, SELFIES, International Chemical Identifier (InChI), and the like. In other examples, the representation of the chemical material can be a pictorial representation comprising an image of the chemical structure of the chemical material.

Then, at operation 508, the system identifies, by the agent, a plurality of models configured to execute the technique for performing the computational task. For instance, the plurality of models can include a machine learning model, a transformer-based model, and a causal model.

Subsequently, at operation 510, the system receives a selection of a model from the plurality of models 508. As discussed above, the selection can be generated by the agent based on an analysis of the plurality of models. That is, by utilizing the strong contextual capabilities of large language models, the agent can determine the applicability of each model in the context of the computational task given the technical benefits and draw backs of each model. Conversely, the selection of the model can be provided by an end user.

Next, at operation 512, the system determines whether the representation of the chemical material is compatible with the model. For example, a given model may only support textual representations of chemical materials. That is, the architecture of the model operates on pictorial representations and thus assumes an input that is formatted as an image. As such, a textual representation of the chemical material can be considered incompatible with the model.

If the representation of the chemical material is compatible with the model, the process 500 proceeds to operation 514 in which the system provides the representation of the chemical material, the molecular descriptor, and the external variable to the model. In one example, the representation of the chemical material, the molecular descriptor, and the external variable are extracted from the input dataset by the agent and provided to the model. In an alternative example, the model accesses the input dataset without first passing through the agent.

Finally, at operation 516, the system invokes, by the agent, a functionality of the model to execute the computational task, thereby providing a response to the prompt. In various examples, the response to the prompt is a model output satisfying the goal described by the prompt. In a specific example, the prompt instructs the system to predict the solubility of a chemical material. Accordingly, the response is a numerical value expressed as the mass of solute per volume of solvent (e.g., g/ml).

If the representation of the chemical material is not compatible with the model, the process 500 proceeds to operation 518 in which the system determines a type of the representation of the chemical material that is compatible with the model. In a specific example, the agent can analyze documentation of the model to determine supported input formats (e.g., a text string input, a pictorial input).

Accordingly, at operation 520 the system converts, by the data processing tool under control of the agent, the data identifying the chemical to the representation of the chemical material. In a specific example, the agent returns to the input dataset to generate a new representation of the chemical material in a format that is compatible with the model. In an alternative example, the existing representation of the chemical material that is not compatible is converted to a format that is compatible with the model. For instance, a pictorial representation is converted into a textual representation.

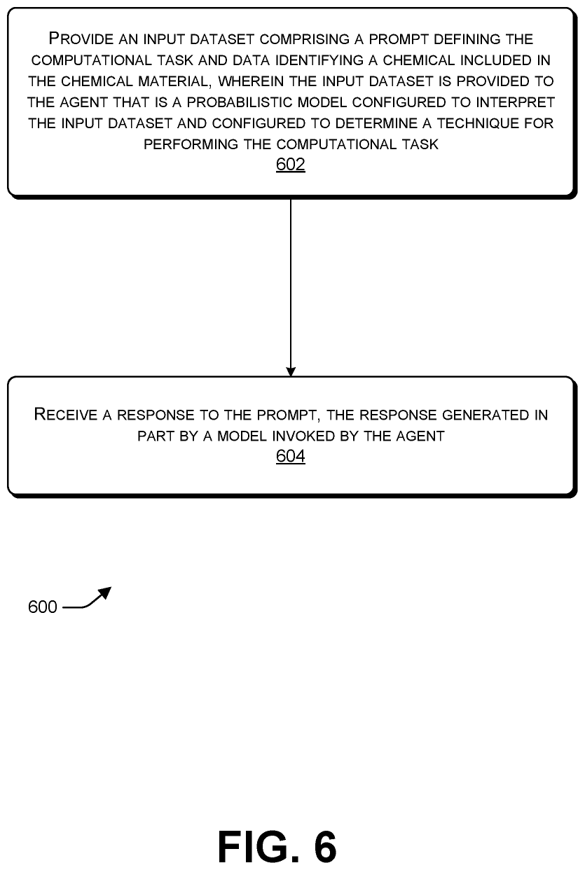

FIG. 6 illustrates a process 600 for interacting with an agent to perform a computational task on a representation of a chemical material. The process 600 begins at operation 602 in which an entity provides an input dataset comprising a prompt defining the computational task and data identifying a chemical included in the chemical material. In one example, the input dataset is provided to the agent that is a probabilistic model configured to interpret the input dataset and configured to determine a technique for performing the computational task.

Then, at operation 604, the entity receives a response to the prompt. In various examples, the response is generated in part by a model invoked by the agent. As described above, the agent can analyze the input dataset to derive the context and technical requirements of a computational task. Accordingly, the agent can invoke a model that is most suitable to the context and technical requirements of the computational task.

For ease of understanding, the processes discussed in this disclosure are delineated as separate operations represented as independent blocks. However, these separately delineated operations should not be construed as necessarily order dependent in their performance. The order in which the process is described is not intended to be construed as a limitation, and any number of the described process blocks may be combined in any order to implement the process or an alternate process. Moreover, it is also possible that one or more of the provided operations is modified or omitted.

The particular implementation of the technologies disclosed herein is a matter of choice dependent on the performance and other requirements of a computing device. Accordingly, the logical operations described herein are referred to variously as states, operations, structural devices, acts, or modules. These states, operations, structural devices, acts, and modules can be implemented in hardware, software, firmware, in special-purpose digital logic, and any combination thereof. It should be appreciated that more or fewer operations can be performed than shown in the figures and described herein. These operations can also be performed in a different order than those described herein.

It also should be understood that the illustrated methods can end at any time and need not be performed in their entireties. Some or all operations of the methods, and/or substantially equivalent operations, can be performed by execution of computer-readable instructions included on a computer-storage media, as defined below. The term “computer-readable instructions,” and variants thereof, as used in the description and claims, is used expansively herein to include routines, applications, application modules, program modules, programs, components, data structures, algorithms, and the like. Computer-readable instructions can be implemented on various system configurations, including single-processor or multiprocessor systems, minicomputers, mainframe computers, personal computers, hand-held computing devices, microprocessor-based, programmable consumer electronics, combinations thereof, and the like.

Thus, it should be appreciated that the logical operations described herein are implemented (1) as a sequence of computer implemented acts or program modules running on a computing system and/or (2) as interconnected machine logic circuits or circuit modules within the computing system. The implementation is a matter of choice dependent on the performance and other requirements of the computing system. Accordingly, the logical operations described herein are referred to variously as states, operations, structural devices, acts, or modules. These operations, structural devices, acts, and modules may be implemented in software, in firmware, in special purpose digital logic, and any combination thereof.

For example, the operations of the processes 500 and 600 can be implemented, at least in part, by modules running the features disclosed herein can be a dynamically linked library (DLL), a statically linked library, functionality produced by an application programing interface (API), a compiled program, an interpreted program, a script, or any other executable set of instructions. Data can be stored in a data structure in one or more memory components. Data can be retrieved from the data structure by addressing links or references to the data structure.

Although the illustration may refer to the components of the figures, it should be appreciated that the operations of the processes 500 and 600 may also be implemented in other ways. In addition, one or more of the operations of the processes 500 and 600 may alternatively or additionally be implemented, at least in part, by a chipset working alone or in conjunction with other software modules. In the example described below, one or more modules of a computing system can receive and/or process the data disclosed herein. Any service, circuit, or application suitable for providing the techniques disclosed herein can be used in operations described herein.

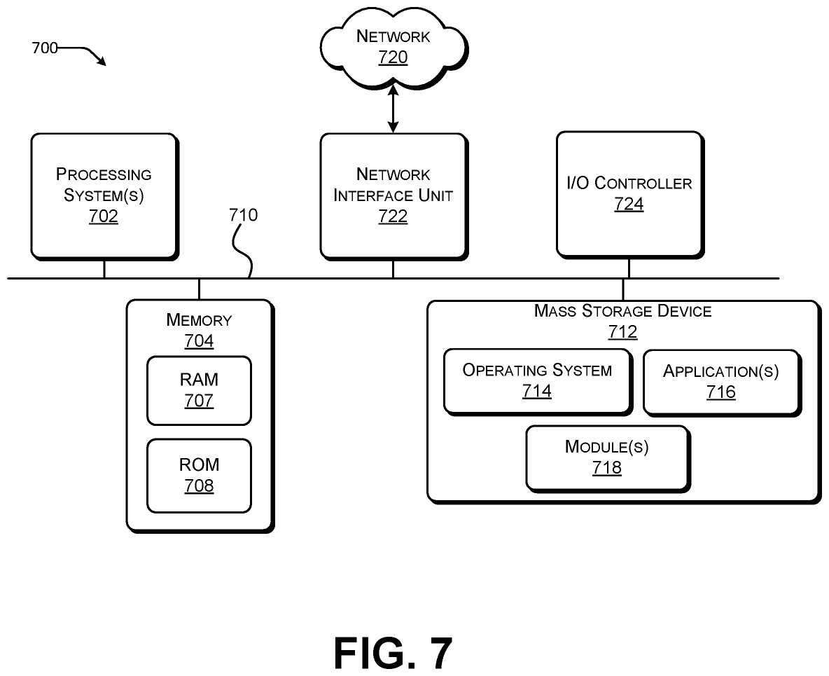

FIG. 7 shows additional details of an example computer architecture 700 for a device, such as a computer or a server configured as part of the system 100, capable of executing computer instructions (e.g., a module or a program component described herein). The computer architecture 700 illustrated in FIG. 7 includes processing system 702, a system memory 704, including a random-access memory 706 (RAM) and a read-only memory (ROM) 708, and a system bus 710 that couples the memory 704 to the processing system 702. The processing system 702 comprises processing unit(s). In various examples, the processing unit(s) of the processing system 702 are distributed. Stated another way, one processing unit of the processing system 702 may be located in a first location (e.g., a rack within a datacenter) while another processing unit of the processing system 702 is located in a second location separate from the first location. For example, the processing system 702 can include graphical processing units (GPUs) for executing complex artificial intelligence applications such as large language models. Moreover, the systems discussed herein can be provided as a distributed computing system such as a cloud service.

Processing unit(s), such as processing unit(s) of processing system 702, can represent, for example, a CPU-type processing unit, a GPU-type processing unit, a field-programmable gate array (FPGA), another class of digital signal processor (DSP), or other hardware logic components that may, in some instances, be driven by a CPU. For example, illustrative types of hardware logic components that can be used include Application-Specific Integrated Circuits (ASICs), Application-Specific Standard Products (ASSPs), System-on-a-Chip Systems (SOCs), Complex Programmable Logic Devices (CPLDs), and the like.

A basic input/output system containing the basic routines that help to transfer information between elements within the computer architecture 700, such as during startup, is stored in the ROM 708. The computer architecture 700 further includes a mass storage device 712 on which an operating system 714, application(s) 716, modules 718, and other data described herein is encoded.

The mass storage device 712 is connected to processing system 702 through a mass storage controller connected to the bus 710. The mass storage device 712 and its associated computer-readable media provide non-volatile storage for the computer architecture 700. Although the description of computer-readable media contained herein refers to a mass storage device, the computer-readable media can be any available computer-readable storage media or communication media that can be accessed by the computer architecture 700.

Computer-readable media includes computer-readable storage media and/or communication media. Computer-readable storage media includes one or more of volatile memory, nonvolatile memory, and/or other persistent and/or auxiliary computer storage media, removable and non-removable computer storage media implemented in any method or technology for encoding of information such as computer-readable instructions, data structures, program modules, or other data. Thus, computer storage media includes tangible and/or physical forms of media included in a device and/or hardware component that is part of a device or external to a device, including RAM, static RAM (SRAM), dynamic RAM (DRAM), phase change memory (PCM), ROM, erasable programmable ROM (EPROM), electrically EPROM (EEPROM), flash memory, compact disc read-only memory (CD-ROM), digital versatile disks (DVDs), optical cards or other optical storage media, magnetic cassettes, magnetic tape, magnetic disk storage, magnetic cards or other magnetic storage devices or media, solid-state memory devices, storage arrays, network attached storage, storage area networks, hosted computer storage or any other storage memory, storage device, and/or storage medium that can be used to store and maintain information for access by a computing device.

In contrast to computer-readable storage media, communication media can embody computer-readable instructions, data structures, program modules, or other data in a modulated data signal, such as a carrier wave, or other transmission mechanism. As defined herein, computer storage media does not include communication media. That is, computer-readable storage media does not include communications media consisting solely of a modulated data signal, a carrier wave, or a propagated signal, per se.

According to various configurations, the computer architecture 700 may operate in a networked environment using logical connections to remote computers through the network 720. The computer architecture 700 may connect to the network 720 through a network interface unit 722 connected to the bus 710. The computer architecture 700 also may include an input/output controller 724 for receiving and processing input from a number of other devices, including a key board, mouse, touch, or electronic stylus or pen. Similarly, the input/output controller 724 may provide output to a display screen, a printer, or other type of output device.

The software components described herein may, when loaded into the processing system 702 and executed, transform the processing system 702 and the overall computer architecture 700 from a general-purpose computing system into a special-purpose computing system customized to facilitate the functionality presented herein. The processing system 702 may be constructed from any number of transistors or other discrete circuit elements, which may individually or collectively assume any number of states. More specifically, the processing system 702 may operate as a finite-state machine, in response to executable instructions contained within the software modules disclosed herein and encoded on the mass storage device 712. These computer-executable instructions may transform the processing system 702 by specifying how the processing system 702 transition between states, thereby transforming the transistors or other discrete hardware elements constituting the processing system 702.

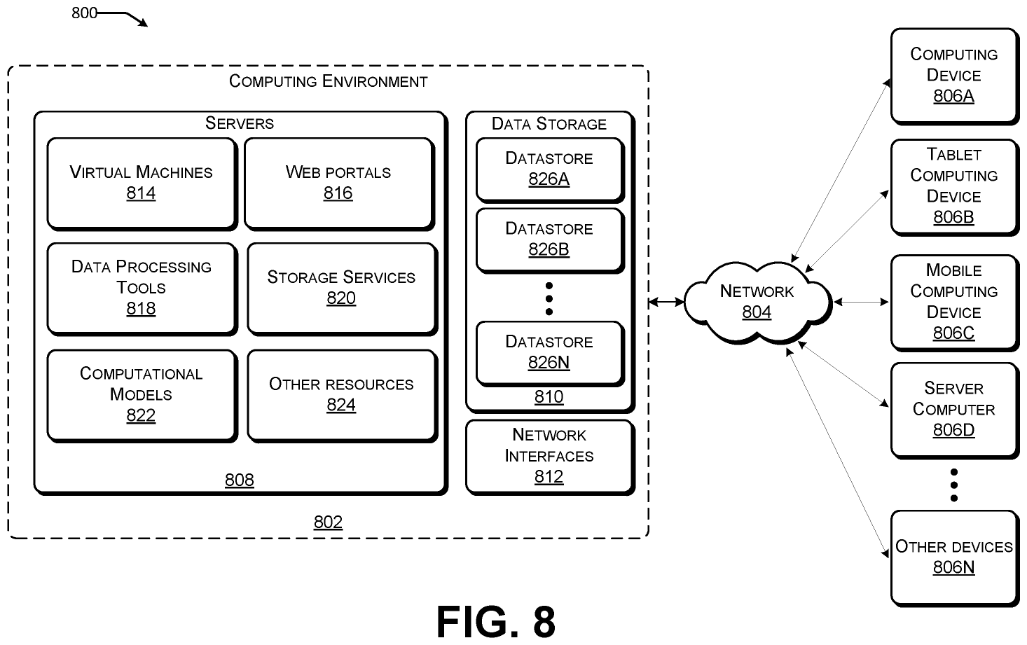

FIG. 8 depicts an illustrative distributed computing environment 800 capable of executing the software components described herein. Thus, the distributed computing environment 800 illustrated in FIG. 8 can be utilized to execute any aspects of the software components presented herein. For example, the distributed computing environment 800 can be utilized to execute aspects of the software components described herein.

Accordingly, the distributed computing environment 800 can include a computing environment 802 operating on, in communication with, or as part of the network 804. The network 804 can include various access networks. One or more client devices 806A-806N (hereinafter referred to collectively and/or generically as “computing devices 806”) can communicate with the computing environment 802 via the network 804. In one illustrated configuration, the computing devices 806 include a computing device 806A such as a laptop computer, a desktop computer, or other computing device: a slate or tablet computing device (“tablet computing device”) 806B: a mobile computing device 806C such as a mobile telephone, a smart phone, or other mobile computing device: a server computer 806D; and/or other devices 806N. It should be understood that any number of computing devices 806 can communicate with the computing environment 802.

In various examples, the computing environment 802 includes servers 808, data storage 610, and one or more network interfaces 812. The servers 808 can host various services, virtual machines, portals, and/or other resources. In the illustrated configuration, the servers 808 host virtual machines 814, Web portals 816, data processing tools 818, storage services 820, and/or computational models 822. As shown in FIG. 8 the servers 808 also can host other services, applications, portals, and/or other resources (“other resources”) 824.

As mentioned above, the computing environment 802 can include the data storage 810. According to various implementations, the functionality of the data storage 810 is provided by one or more databases operating on, or in communication with, the network 804. The functionality of the data storage 810 can also be provided by one or more servers configured to host data for the computing environment 800. The data storage 810 can include, host, or provide one or more real or virtual datastores 826A-826N (hereinafter referred to collectively and/or generically as “datastores 826”). The datastores 826 are configured to host data used or created by the servers 808 and/or other data. That is, the datastores 826 also can host or store web page documents, word documents, presentation documents, data structures, algorithms for execution by a recommendation engine, and/or other data utilized by any application program. Aspects of the datastores 826 may be associated with a service for storing files.

The computing environment 802 can communicate with, or be accessed by, the network interfaces 812. The network interfaces 812 can include various types of network hardware and software for supporting communications between two or more computing devices including the computing devices and the servers. It should be appreciated that the network interfaces 812 also may be utilized to connect to other types of networks and/or computer systems.

It should be understood that the distributed computing environment 800 described herein can provide any aspects of the software elements described herein with any number of virtual computing resources and/or other distributed computing functionality that can be configured to execute any aspects of the software components disclosed herein. According to various implementations of the concepts and technologies disclosed herein, the distributed computing environment 800 provides the software functionality described herein as a service to the computing devices. It should be understood that the computing devices can include real or virtual machines including server computers, web servers, personal computers, mobile computing devices, smart phones, and/or other devices. As such, various configurations of the concepts and technologies disclosed herein enable any device configured to access the distributed computing environment 800 to utilize the functionality described herein for providing the techniques disclosed herein, among other aspects.

The following example clauses describe multiple possible embodiments for implementing the features described in this disclosure. The various embodiments described herein are not limiting nor is every feature of any given embodiment required to be present in another embodiment. Any two or more of the embodiments may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Example Clause A, a method for interacting with an agent to perform a computational task on a representation of a chemical material, the method comprising: receiving an input dataset comprising a prompt defining the computational task, data identifying a chemical included in the chemical material, a molecular descriptor describing a characteristic of the chemical, and an external variable: interpreting the input dataset by the agent that is a probabilistic model configured to determine a technique for performing the computational task: generating, by a data processing tool under control of the agent, the representation of the chemical material: identifying, by the agent, a plurality of models configured to execute the technique for performing the computational task: receiving a selection of a model from the plurality of models: providing the representation of the chemical material, the molecular descriptor, and the external variable to the model; and invoking, by the agent, a functionality of the model to execute the computational task thereby providing a response to the prompt.

Example Clause B, the method of Example Clause A, wherein receiving the input dataset comprises extracting, by the agent, the molecular descriptor from a chemical database.

Example Clause C, the method of Example Clause A or Example Clause B, further comprising: determining a type of the representation of the chemical material that is compatible with the model; and converting, by the data processing tool under control of the agent, the data identifying the chemical to the representation of the chemical material.

Example Clause D, the method of any one of Example Clause A through C, wherein the representation of the chemical material comprises a text string representation.

Example Clause E, the method of any one of Example Clause A through D, wherein the plurality of models identified by the agent include at least one of a machine learning model, a transformer-based model, or a causal model.

Example Clause F, the method of any one of Example Clause A through D, wherein receiving the selection of the model comprises: calculating an applicability factor for each of the plurality of models based on the input dataset, wherein the applicability factor quantifies an ability of each of the plurality of models to execute the technique for performing the computational task; and selecting, by the agent, the model from the plurality of models based on the applicability factor calculated for each of the plurality of models.

Example Clause G, the method of Example Clause F, wherein the applicability factor is calculated based on a characteristic of the input dataset and a characteristic of the computational task.

Example Clause H, the method of Example Clause F, wherein the chemical material comprises two or more chemicals: the computational task comprises identifying a chemical property of the chemical material; and the method further comprises training the model to predict the chemical property of the chemical material.

Example Clause I, a system comprising: a processing system: a computer-readable medium having encoded thereon instructions executable by the processing system; a data input module configured to receive an input dataset comprising a prompt defining a computational task to be performed on a representation of a chemical material, data identifying a chemical included in the chemical material, a molecular descriptor describing a characteristic of the chemical, and an external variable: an agent configured to interpret the input dataset to determine a technique for performing the computational task, wherein the technique comprises a chain of calls to a data processing tool and a model: the data processing tool configured by the agent to generate a representation of the chemical material: a pre-selection module configured by the agent to identify a plurality of models, wherein each of the plurality of models is capable of being configured by the agent to execute the technique for performing the computational task; and an output module configured to provide a response to the prompt following an execution of the technique for performing the computational task, wherein the response comprises a requested output derived from the input dataset by a model selected from the plurality of models.

Example Clause J, the system of Example Clause I, wherein the molecular descriptor in the input dataset is extracted from a chemical database by the data input module under control of the agent.

Example Clause K, the system of Example Clause I or Example Clause J, wherein the agent is further configured to determine a type of the representation of the chemical material that is compatible with the model; and the data processing tool is further configured, under control of the agent, to convert the data identifying the chemical to the type of representation of the chemical material that is compatible with the model.

Example Clause L, the system of any one of Example Clause I through K, wherein the plurality of models identified by the agent comprises at least one of a machine learning model, a transformer-based model, or a causal model.

Example Clause M, the system of any one of Example Clause I through L, wherein: the pre-selection module is further configured, under control of the agent, to calculate an applicability factor for each of the plurality of models based on the input dataset, wherein the applicability factor quantifies an ability of each of the plurality of models to execute the technique for performing the computational task; and the agent is further configured to select the model from the plurality of models based on the applicability factor calculated for each of the plurality of models.

Example Clause N, the system of any one of Example Clause I through M further comprising a second model, wherein the agent is further configured to train the model and the second model, and the computational task is performed in part using the model and in part using the second model.

Example Clause O, a method for interacting with an agent to perform a computational task on a representation of a chemical material, the method comprising: providing an input dataset comprising a prompt defining the computational task and data identifying a chemical included in the chemical material, wherein the input dataset is provided to the agent that is a probabilistic model configured to interpret the input dataset and configured to determine a technique for performing the computational task; and receiving a response to the prompt, the response generated in part by a model invoked by the agent.

Example Clause P, the method of Example Clause O, wherein the input dataset further comprises a molecular descriptor extracted from a chemical database responsive to the data identifying the chemical.

Example Clause Q, the method of Example Clause O or Example Clause P, wherein the agent is further configured to: determine a type of the representation of the chemical material that is compatible with the model; and cause a data processing tool to convert the data identifying the chemical to the representation of the chemical material.

Example Clause R, the method of any one of Example Clause O through Example Clause Q, wherein the technique for performing the computational task comprises invoking, by the agent, a chain of calls to a data processing tool and the model, wherein the model is one of a machine learning model, a transformer-based model, or a causal model.

Example Clause S, the method of any one of Example Clause O through Example Clause R, further comprising: selecting a model from a plurality of models identified by the agent that are each configured to perform at least part of the computational task, wherein the selecting of the model causes the agent to invoke a functionality of the model to execute the technique for performing the computational task.

Example Clause T, the method of Example Clause S, wherein the receiving the response comprises: receiving a display of a ranked list generated by the agent based on an ability of each of the plurality of models to execute the technique for performing the computational task; and providing an input indicating a selection of a model from the ranked list, the input causing the agent to invoke the model indicated by the input.

Conditional language such as, among others, “can,” “could,” “might” or “may,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example. Conjunctive language such as the phrase “at least one of X, Y or Z,” unless specifically stated otherwise, is to be understood to present that an item, term, etc. may be either X, Y, or Z, or a combination thereof.

The terms “a,” “an,” “the” and similar referents used in the context of describing the invention (especially in the context of the following claims) are to be construed to cover both the singular and the plural unless otherwise indicated herein or clearly contradicted by context. The terms “based on,” “based upon,” and similar referents are to be construed as meaning “based at least in part” which includes being “based in part” and “based in whole” unless otherwise indicated or clearly contradicted by context.

In addition, any reference to “first,” “second,” etc. elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different models).

In closing, although the various configurations have been described in language specific to structural features and/or methodological acts, it is to be understood that the subject matter defined in the appended representations is not necessarily limited to the specific features or acts described. Rather, the specific features and acts are disclosed as example forms of implementing the claimed subject matter.

1. A method for interacting with an agent to perform a computational task on a representation of a chemical material, the method comprising:
receiving an input dataset comprising a prompt defining the computational task, data identifying a chemical included in the chemical material, a molecular descriptor describing a characteristic of the chemical, and an external variable;
interpreting the input dataset by the agent that is a probabilistic model configured to determine a technique for performing the computational task;
generating, by a data processing tool under control of the agent, the representation of the chemical material;
identifying, by the agent, a plurality of models configured to execute the technique for performing the computational task;
receiving a selection of a model from the plurality of models;
providing the representation of the chemical material, the molecular descriptor, and the external variable to the model; and
invoking, by the agent, a functionality of the model to execute the computational task thereby providing a response to the prompt.

2. The method of claim 1, wherein receiving the input dataset comprises extracting, by the agent, the molecular descriptor from a chemical database.

3. The method of claim 1, further comprising:
determining a type of the representation of the chemical material that is compatible with the model; and
converting, by the data processing tool under control of the agent, the data identifying the chemical to the representation of the chemical material.

4. The method of claim 1, wherein the representation of the chemical material comprises a text string representation.

5. The method of claim 1, wherein the plurality of models identified by the agent include at least one of a machine learning model, a transformer-based model, or a causal model.

6. The method of claim 1, wherein receiving the selection of the model comprises:
calculating an applicability factor for each of the plurality of models based on the input dataset, wherein the applicability factor quantifies an ability of each of the plurality of models to execute the technique for performing the computational task; and
selecting, by the agent, the model from the plurality of models based on the applicability factor calculated for each of the plurality of models.

7. The method of claim 6, wherein the applicability factor is calculated based on a characteristic of the input dataset and a characteristic of the computational task.

8. The method of claim 1, wherein the chemical material comprises two or more chemicals;
the computational task comprises identifying a chemical property of the chemical material; and
the method further comprises training the model to predict the chemical property of the chemical material.

9. A system comprising:
a processing system;
a computer-readable medium having encoded thereon instructions executable by the processing system;
a data input module configured to receive an input dataset comprising a prompt defining a computational task to be performed on a representation of a chemical material, data identifying a chemical included in the chemical material, a molecular descriptor describing a characteristic of the chemical, and an external variable;
an agent configured to interpret the input dataset to determine a technique for performing the computational task, wherein the technique comprises a chain of calls to a data processing tool and a model;
the data processing tool configured by the agent to generate a representation of the chemical material;
a pre-selection module configured by the agent to identify a plurality of models, wherein each of the plurality of models is capable of being configured by the agent to execute the technique for performing the computational task; and
an output module configured to provide a response to the prompt following an execution of the technique for performing the computational task, wherein the response comprises a requested output derived from the input dataset by a model selected from the plurality of models.

10. The system of claim 9, wherein the molecular descriptor in the input dataset is extracted from a chemical database by the data input module under control of the agent.

11. The system of claim 9, wherein the agent is further configured to determine a type of the representation of the chemical material that is compatible with the model; and
the data processing tool is further configured, under control of the agent, to convert the data identifying the chemical to the type of representation of the chemical material that is compatible with the model.

12. The system of claim 9, wherein the plurality of models identified by the agent comprises at least one of a machine learning model, a transformer-based model, or a causal model.

13. The system of claim 9, wherein:
the pre-selection module is further configured, under control of the agent, to calculate an applicability factor for each of the plurality of models based on the input dataset, wherein the applicability factor quantifies an ability of each of the plurality of models to execute the technique for performing the computational task; and
the agent is further configured to select the model from the plurality of models based on the applicability factor calculated for each of the plurality of models.

14. The system of claim 9 further comprising a second model, wherein the agent is further configured to train the model and the second model, and the computational task is performed in part using the model and in part using the second model.

15. A method for interacting with an agent to perform a computational task on a representation of a chemical material, the method comprising:
providing an input dataset comprising a prompt defining the computational task and data identifying a chemical included in the chemical material, wherein the input dataset is provided to the agent that is a probabilistic model configured to interpret the input dataset and configured to determine a technique for performing the computational task; and
receiving a response to the prompt, the response generated in part by a model invoked by the agent.

16. The method of claim 15, wherein the input dataset further comprises a molecular descriptor extracted from a chemical database responsive to the data identifying the chemical.

17. The method of claim 15, wherein the agent is further configured to:
determine a type of the representation of the chemical material that is compatible with the model; and
cause a data processing tool to convert the data identifying the chemical to the representation of the chemical material.

18. The method of claim 15, wherein the technique for performing the computational task comprises invoking, by the agent, a chain of calls to a data processing tool and the model, wherein the model is one of a machine learning model, a transformer-based model, or a causal model.

19. The method of claim 15, further comprising:
selecting a model from a plurality of models identified by the agent that are each configured to perform at least part of the computational task, wherein the selecting of the model causes the agent to invoke a functionality of the model to execute the technique for performing the computational task.

20. The method of claim 19, wherein the receiving the response comprises:
receiving a display of a ranked list generated by the agent based on an ability of each of the plurality of models to execute the technique for performing the computational task; and
providing an input indicating a selection of a model from the ranked list, the input causing the agent to invoke the model indicated by the input.
